(19) United States
(12) Patent Application Publication
(10) Pub. No.: US 2014/0274780 A1
(43) Pub. Date: Sep. 18, 2014

(54) METHODS OF IMPROVING SURVIVAL IN CANCER

(71) Applicant: PSertain Technologies, Maplewood, NJ (US)

(72) Inventor: Gregory Miles, Hillsborough, NJ (US)

(21) Appl. No.: 14/214,640

(22) Filed: Mar. 14, 2014

Related U.S. Application Data
(60) Provisional application No. 61/800,788, filed on Mar. 15, 2013.

(51) Int. Cl. C12Q 1/68

(52) U.S. Cl. CPC C12Q 1/6886
USPC 506/9; 435/6.12; 536/23.1; 506/16

(57) ABSTRACT

Methods of improving cancer therapy outcomes are provided. Diagnostics useful for evaluating patients based on microRNA signatures of cancer tissue are provided.

This application claims priority under 35 U.S.C. §119(e) to provisional application Ser. No. 60/800,788 filed on Mar. 15, 2013, the entire disclosure of which is hereby expressly incorporated by reference.

### BACKGROUND

A difficulty in the treatment of cancer is assessing the potential of a patient to respond to a particular cancer therapy. In most cases, a patient is treated with an initial therapy and their degree of response is observed empirically. Based on the patient's response to the therapy, the patient continues on the therapy or is switched to another therapy. In cases where a patient does not respond positively to the initial therapy, valuable treatment time is lost. The present invention addresses this problem by providing diagnostics combined with treatment regimens to indicate the most appropriate initial therapy for a patient.

MicroRNAs are known to be associated with aggressive or poor prognosis phenotypes in cancer [1-6]. Drug-resistant cells that remain post-therapy are the primary cause of mortality in cancer. Therefore it follows that certain microRNAs are markers of, or play a role in, cancer response or resistance to various therapies. The present invention identifies specific combinations of microRNAs whose expression is associated degrees of cancer survival. A novel analytical method was employed that compares survival differences with expression levels of these microRNA combinations and identifies expression level “signatures,” associated with degrees of cancer survival when a patient receives a particular therapy.

Algorithms and protocols for diagnostic tests have been reported which predict clinical outcome for a human subject diagnosed with a specific cancer following surgical resection of said cancer. Tests have been implemented for breast cancer and colorectal cancer prognosis using normalized expression levels of RNA transcripts of specific genes within a biological sample comprising of cancer cells obtained from a human subject. From these expression levels, clinical outcome is expressed in terms of one or more of the following: Recurrence Score, Recurrence-Free Interval, Overall Survival, Disease-Free Survival, or Distant Recurrence-Free Interval. See, for example, U.S. Pat. Nos. 8,465,923, 8,273,537, 7,526,387 and 7,569,345.

### BRIEF DESCRIPTION OF THE DRAWINGS

FIG. 1.—Kaplan-Meier survival plot of Class Ov1B. Survival plot of patients (dotted line) from Class Ov1B versus the remaining patients (solid line).

FIG. 2.—Kaplan-Meier survival plot of Class Ov1A. Survival plot of patients (dotted line) from Class Ov1A versus the remaining patients (solid line).

FIG. 3—Kaplan-Meier survival plot of Class Ov2A. Survival plot of patients (dotted line) from Class Ov2A versus the remaining patients (solid line).

FIG. 4—Kaplan-Meier survival plot of Class Ov2B. Survival plot of patients (dotted line) from Class Ov2B versus the remaining patients (solid line).

FIG. 5—Survival plot of Class Ov3. Survival plots of patients (dotted line) from Class Ov3 show significantly poorer prognosis and poor response to cisplatin/carboplatin+taxol chemotherapy compared with the remaining patients (solid line).

FIG. 6—Survival plot of robust poor prognosis signature from independent dataset. Patients (dotted line) from Class Ov3 show significantly poorer prognosis and poor response to cisplatin/carboplatin+taxol chemotherapy compared with the remaining patients (solid line) in an independent dataset.

FIG. 7—Kaplan-Meier survival plot of Class G1A. Survival plot of patients (dotted line) from Class G1A versus the remaining patients (solid line).

FIG. 8—Kaplan-Meier survival plot of Class G2A. Survival plot of patients (dotted line) from Class G2A versus the remaining patients (solid line).

FIG. 9—Kaplan-Meier survival plot of Class G2B. Survival plot of patients (dotted line) from Class G2B versus the remaining patients (solid line).

### DETAILED DESCRIPTION

In this description, a “Class” refers to a group of patients whose tumors can be classified by a “Signature” made up of a specific combination of microRNAs expressed at certain levels. The expression level of the microRNAs constitute the signature associated with a specific prognosis and level of therapeutic response. A “benchmark” refers to the level at which a microRNA must be expressed to fall within a “signature”. When assaying microRNA expression in a tumor from a patient, the determination of whether the expression level falls within the signature is made by comparing the expression level of the microRNA to a benchmark. For some classes, the microRNA expression level is above a benchmark. In other classes, the level of expression must be below the benchmark. When assaying control microRNA expression levels in a tumor from a patient, the control microRNA expression levels must be between the lower and upper boundaries of the benchmark. Thus, a signature is a combination of microRNAs expressed at particular levels.

Ovarian cancer, an extremely deadly disease for which there are greater than 22,000 newly diagnosed cases in the United States each year, is one area of importance. Nearly all of these patients are treated by surgical resection of the tumor followed by an aggressive platinum/taxane chemotherapy regimen. Between 10-15% of patients are non-responsive (recurrence<6 months after treatment) and are considered platinum resistance. The ability to identify responders and non-responders can determine whether a particular patient should receive standard therapies or to proceed to experimental trials. Evaluation of new treatment standards (such as AVASTIN® (Genentech, Inc., San Francisco Calif.), which demonstrates very limited benefits and shows significant toxicity) may also be assisted by prospective identification of patients with tumors resistant to platinum/taxane chemotherapy.

The present invention address these problems by identifying novel combinations of microRNAs expressed at certain levels referred to herein as signatures. Certain signatures are indicative of an increase in survival of patients with ovarian cancer when the patient is provided with a particular treatment. The present invention, in certain embodiments, identifies specific microRNA signatures which most robustly define classes of patients with similar response to therapy.

The present invention, in certain embodiments, also identifies an algorithm which assigns the patient a PScore (Prognosis Score) for each microRNA signature. The PScore indicates whether or not the microRNA expression levels fall within the range which describes the signature. The term “norm factor”, shorthand for normalization factor, will refer to a microRNA-specific value that is used to normalize tumor-assayed microRNA expression values. On the ensuing tables, “Norm Factor 1” defines a unique numerical quantity that will be subtracted from the assayed expression level for the specific microRNA that it refers to. On the ensuing tables, “Norm Factor 2” defines a numerical quantity that will be divided from the expression level obtained after the use of Norm Factor 1. The final normalized expression value for a single microRNA will be defined as: (Assayed expression−Norm Factor 1)/Norm Factor 2.

Based on these findings, it has been determined and bioinformatically validated, in an independent dataset, that microRNA signatures can strongly indicate a reduced or enhanced prognosis in patients with ovarian cancer when treated with platinum based therapies.

Glioblastoma multiforme is another extremely aggressive and deadly form of cancer. In order to facilitate treating patients with appropriate therapies, this invention identifies microRNA signatures correlated with survival differences and response in glioblastoma when a patient is provided with a particular therapy. This invention identifies specific microRNA signatures which are predictive of survival and response in combination with a therapy. The present invention, in certain embodiments, also identifies an algorithm which assigns a PScore described previously that indicates whether a signature is exhibited in a tumor from a patient.

In one embodiment of this invention, four distinct survival-based Classes of microRNAs are identified that are indicative of cisplatin/carboplatin+taxol response in ovarian cancer patients. The microRNA expression-based signatures associated with these Classes define unique patient sets, including: a) Poor-survival Classes when certain microRNAs are over-expressed and certain therapy is provided; b) A poor-survival class when specific microRNAs are under-expressed and a particular therapy is provided, and; c) An improved-survival class when certain microRNAs are under-expressed and a particular therapy is provided. The invention further identifies a class, Ov3, with a signature consisting of four microRNAs (hsa-miR-381, hsa-miR-410, hsa-miR-376a, and hsa-miR-377) such that if at least one is over-expressed, patient prognosis is significantly reduced when a particular therapy is provided. The signature for each class also contains distinct control microRNAs for normalization.

The present invention also provides three distinct survival-based classes with signatures of microRNAs expressed in glioblastoma multiforme that are associated with degrees of patient response to therapy with temodol/temozolomide. The microRNA signatures are correlated with both poor and improved survival prognoses when patients from this class are treated with this therapy.

Identifying patients with good prognoses in response to a particular therapy prior to treatment allows for placing the patient on appropriate therapy with an expectation of a positive outcome. Identifying patients with poorer prognosis in relation to a particular therapy prior to treatment allows for more aggressive or alternative initial treatment of their disease. Identification of these patients can also prevent unnecessary treatments in cases where extension of survival is not feasible. The present invention provides diagnostics based on novel combinations of microRNAs and methods of placing patients on appropriate initial therapies.

The present invention also identifies an algorithm which assigns the patient a PScore (Prognosis Score) for each microRNA signature that determines whether or not their expression levels fall within the signature “benchmarks”. The PScore is described in the example below.

### Example 1

### Calculation of PScore

The present invention, as previously stated, discloses an algorithm that is used to determine, for each patient, a PScore for each Class. Each microRNA from a Class signature will have its own “subscore” consisting of a single binary value (1 or 0). If the expression of the specific microRNA is, depending on the Class, over, under, or within the range of its benchmark, a binary value of 1 is given. If the expression of the specific microRNA is not, depending on the Class, over, under, or within the range of its benchmark, a binary value of 0 is given. Each PScore is compiled by taking the sum of the subscores of the microRNAs from a single class signature and dividing by the number of microRNAs within the signature of the class. A PScore greater than or equal to 0.5 indicates that the patient is a member of that specific class and has a tumor that exhibits the signature of its class. A subscore less than 0.5 indicates that the patient is not a member of that specific class and does not exhibit the signature associated with it.

Each microRNA subscore is computed as the following: Baseline ranges for the assay must first be established using positive and negative controls. The negative control will establish the “zero” point, while the positive control shall establish the highest level of expression for the instrument. In order to compare the assayed values with the established standards outlined in Tables 1 and 2, the controls will be normalized to benchmark control values established for each class. For Class Ov1A, the assayed negative control will be normalized to a benchmark of −3.228492444 and the assayed positive control will be normalized to a benchmark of 12.82602161. For Class Ov1B, the assayed negative control will be normalized to a benchmark of −4.256095457 and the assayed positive control will be normalized to a benchmark of 11.7905749. For Class Ov2A, the assayed negative control will be normalized to a benchmark of −4.284986418 and the assayed positive control will be normalized to a benchmark of 12.33382845. For Class Ov2B, the assayed negative control will be normalized to a benchmark of −4.68141474 and the assayed positive control will be normalized to a benchmark of 11.93899365.

A normalized expression level is obtained by taking the assayed expression level measured for a specific microRNA and first subtracting its unique “Norm factor 1” component. This result is then divides by its unique “Norm factor 2” component. All control microRNAs from a Class must have normalized expression values within the “benchmark” range specified in Table 1 or Table 2 in order for the patient to be considered for that Class. Finally, for Classes Ov1A and Ov1B, the subscore of a microRNA is a “1” if the normalized assayed expression level is greater than the benchmark specified in Table 1. Should this condition not be fulfilled, the subscore for the microRNA is “0”. For Classes Ov2A and Ov2B, the normalized assayed expression level of a microRNA must be lower than the benchmark specified in Table 2 to attain a subscore of “1”. Should this condition not be fulfilled, the subscore for the microRNA is “0”.

Additionally, the present invention discloses a Class Ov3 comprising of four distinct microRNAs whose expression level constitute its signature: hsa-miR-381, hsa-miR-376a, hsa-miR-410, and hsa-miR-377, such that at least one exhibits an elevated level of expression that exceed the benchmarks specified in Table 2 in tumors of patients with poorer prognosis on cisplatin/carboplatin plus taxol therapy. Patients with tumors exhibiting this signature should receive more aggressive initial therapy. This signature, further confirmed and bioinformatically validated within an independent dataset, provides evidence of a signature of microRNAs that describe a class of patients and predicts poor patient response.

### Example 2

### MicroRNAs and Improvement of Survival in Ovarian Cancer

In the performance of an assay, the experimenter obtains tissue from fresh frozen or FFPE primary tumors from serous ovarian cancer patients who are to be treated with cisplatin/carboplatin plus taxol chemotherapy. Signature and control MicroRNAs are extracted using a small RNA extraction kit, e.g. RNAEASY, or other appropriate methods, Expression is quantified using a method such as qRTPCR, microarray hybridization, next generation sequencing technologies, or flow cytometer.

The analysis yielded distinct classes along with five unique control microRNAs: Class Ov1B, whose signature consists of five microRNAs, and Class Ov1A, whose signature consists of eleven microRNAs (Table. 1). FIG. 1 depicts the survival plot of patients with tumors that are described by the signature of Class Ov1B (dotted line) compared with the remaining patient tumor samples (solid line). The significant separation defines a distinct class of patients with poorer prognosis and therefore poor response to cisplatin/carboplatin plus taxol chemotherapy, indicated by the presence of the signature, i.e., an elevated level of expression of the microRNAs which exceeds the benchmark (specified in Table 1) of the microRNAs of Class Ov1B. The low p-value (0.0084) indicates that these five microRNAs are up-regulated in patients with poorer prognosis with this therapy.

FIG. 2 demonstrates a similar plot comparing survival of patients having tumors (dotted line) that are described by the signature of Class Ov1A, which consists of eleven microRNAs, versus the remaining patients tumor samples (solid line). The significant separation defines a distinct class of patients with poorer prognosis and therefore poor response to cisplatin/carboplatin plus taxol chemotherapy, indicated by an elevated level of expression which exceeds the benchmark (specified in Table 1) of the microRNA of Class Ov1A. The p-value is again low (0.0008), indicating that these microRNAs are up-regulated in tumors from patients with a poorer prognosis.

Further analysis generated two additional classes: Class Ov2A, whose signature is represented by the expression levels of eight microRNAs, and Class Ov2B, whose signature is represented by the expression levels of nine microRNAs. FIG. 3 depicts the survival plot of patients with tumors that are described by the signature of Class Ov2A (dotted line) versus the remaining patients' tumor samples (solid line). The significant separation defines a distinct class of patients with improved prognosis and a positive response to cisplatin/carboplatin plus taxol chemotherapy as indicated by a reduced level of expression which falls below the benchmark (specified in Table 2) of the microRNA of Class Ov2A. The low p-value (0.0362) indicates that these eight microRNAs were down-regulated in tumors from patients with improved prognosis.

FIG. 4 shows a similar plot comparing survival of patients with tumors (dotted line) that are described by the signature of Class Ov2B (consisting of nine microRNAs) with the remaining patients' tumor samples (solid line). The significant separation defines a distinct class of patients with poor prognosis and therefore poor response to cisplatin/carboplatin plus taxol chemotherapy. This is indicated by a reduced level of expression which falls below the benchmark (specified in Table 2) of the microRNA of Class Ov2B in the tumors of these patients. The p-value is again low (0.0092), indicating that down-regulation of these microRNAs is present in tumors from patients with poor prognosis.

Table 1 below lists the signature microRNAs from Classes Ov1A and Ov1B. Table 2 similarly lists the signature microRNAs from Classes Ov2A and Ov2B. Additional analysis confirmed a more robust set of three microRNAs (bolded Table 1) from Class Ov1B, hsa-miR-381, hsa-miR-376a, and hsa-miR-377, and one microRNA from Ov2A, hsa-mir-410 (bolded in Table 2), whose expression at particular levels constitutes a signature for an additional class, Class Ov3, indicative of poor prognosis.

FIG. 5 depicts a survival curve of patients with tumors (dotted line) in which at least one of the above Class Ov3 four microRNAs is over-expressed compared to the benchmark level. The significant separation defines a unique signature of poor prognosis and response that was confirmed within an independent dataset. The strong p-value (0.0013) provides evidence of a poor-prognosis microRNA-based signature within this group compared with the remaining patients (solid line). FIG. 6 further confirms this poor-prognosis class within an independent dataset. The significant separation confirms the predictive signature of poor prognosis outlined in FIG. 5. The strong p-value (0.0149) validates this signature.

### Table 1—MicroRNA Classes Predictive of Response to Therapy.

Classes described by microRNA signatures in tumors that are predictive of response to cisplatin/carboplatin plus taxol therapy. Classes Ov1A and Ov1B have a poor prognosis when the microRNAs are elevated above the benchmark and improved prognosis when microRNA expression is lower. For Classes Ov1A and Ov1B, a binary subscore of a microRNA is assigned a score of 1 if the assayed expression level is greater than the benchmark and a score of 0 if the assayed expression level is less than the benchmark. The “norm factors” are specifically calculated numeric representations by which patient data needs to be normalized. “Norm factor 1” shall be subtracted from the patient assayed expression level. Subsequently, this total will be divided by “Norm factor 2” to produce normalized expression values.

TABLE 1

| | miRBase | | | |
|---|---|---|---|---|
| | Accession | | | |
| | (v20) | Benchmark | Norm factor 1 | Norm Factor 2 |
| Class Ov1A | | | | |
| hsa-miR-33b | MI0003646 | 1.228520893 | −4.600513196 | 1.052181498 |
| hsa-miR-30d | MI0000255 | 1.317542631 | 4.97712896 | 0.982415096 |
| hsa-miR-30d* (now | MIMAT0004551 | 1.317827841 | −1.829095946 | 0.996933586 |
| goes by hsa-miR- | | | | |
| 30d-3p) | | | | |
| hsa-miR-370 | MI0000778 | 1.342472815 | −2.180674506 | 1.44731479 |
| hsa-miR-934 | MI0005756 | 1.31240231 | −2.985339555 | 1.711150408 |
| hsa-miR-519e* (now | MIMAT0002828 | 1.240174782 | −3.597639316 | 0.927101135 |
| goes by hsa-miR- | | | | |
| 519e-5p) | | | | |
| hsa-miR-30b* | MIMAT0004589 | 1.361580223 | −1.74298474 | 0.770748215 |
| hsa-miR-663a | MI0003672 | 1.33598847 | 1.692041639 | 1.131280034 |
| hsa-miR-583 | MI0003590 | 1.427842617 | −4.311340825 | 1.143804839 |
| hsa-miR-526b | MI0003150 | 1.269653745 | −3.92330944 | 1.142756826 |
| hsa-miR-9 (control) | MIMAT0000441 | −0.915209972 to | −3.70936744 | 1.876567065 |
| (now goes by hsa- | | −0.052107897 | | |
| miR-9-5p | | | | |
| hsa-miR-9* (control) | MIMAT0000442 | −0.940279383 to | −3.268793578 | 1.783030967 |
| (now goes by hsa- | | −0.166443422 | | |
| miR-9-3p | | | | |
| hsa-miR-501-5p | MIMAT0002872 | −0.1110648 to | −1.171002267 | 0.789464804 |
| (control) | | 0.767432718 | | |
| hsa-miR-488 | MI0003123 | −0.948446804 to | −3.649730855 | 1.767542582 |
| (control) | | −0.019385949 | | |
| hsa-miR-144* | MI0000460 | −1.059332319 to | −3.65013101 | 1.50932409 |
| (control) | | −0.079706005 | | |
| Class Ov1B | | | | |
| hsa-miR-136 | MI0000475 | 1.087306555 | 0.893319952 | 1.236136958 |
| hsa-miR-337-5p | MIMAT0004695 | 1.123569374 | −1.11057922 | 1.15260585 |
| hsa-miR-377 | MI0000785 | 1.088520036 | 0.901390886 | 1.232013177 |
| hsa-miR-381 | MI0000789 | 1.063867896 | 0.013457223 | 1.072079829 |
| hsa-miR-376a-1 | MI0000784 | 1.08557208 | 1.487085661 | 1.250895764 |
| hsa-miR-379 | MI0000787 | 0.71139873 to | −1.164830838 | 1.306786907 |
| (control) | | 1.982465335 | | |
| hsa-miR-411 | MI0003675 | 0.692584115 to | −4.423354025 | 1.068534927 |
| (control) | | 2.011466319 | | |
| hsa-miR-299-3p | MIMAT0000687 | 0.760251382 to | −2.579440147 | 1.311385678 |
| (control) | | 2.322145511 | | |
| hsa-miR-154 | MI0000480 | 0.850251002 to | −1.427770028 | 1.114498727 |
| (control) | | 2.249478771 | | |
| hsa-miR-376c | MI0000776 | 0.811164008 to | 1.632163218 | 1.243353839 |
| (control) | | 2.368833356 | | |

Table 2—Additional microRNA Classes Predictive of Response to Therapy.

Classes described by microRNA signatures which are predictive of response to cisplatin/carboplatin plus taxol therapy. Class Ov2A has a good prognosis when the microRNAs are repressed below the benchmark while Class Ov2B has a poor prognosis when the microRNAs are repressed below the benchmark. For Classes Ov2A and Ov2B, a binary subscore of a microRNA is assigned a score of 1 if the assayed expression level is less than the benchmark and a score of 0 if the assayed expression level is greater than the benchmark. The “norm factors” are specifically calculated numeric representations by which patient data needs to be normalized. “Norm factor 1” shall be subtracted from the patient assayed expression level. Subsequently, this total will be divided by “Norm factor 2” to produce normalized expression values.

TABLE 2

| | miRBase | | | |
|---|---|---|---|---|
| | Accession (v20) | Benchmark | Norm factor 1 | Norm Factor 2 |
| Class Ov2A | | | | |
| hsa-miR-136 | MI0000475 | −1.360236922 | 0.893319952 | 1.236136958 |
| hsa-miR-377 | MI0000785 | −1.396727531 | 0.901390886 | 1.232013177 |
| hsa-miR-410 | MI0002465 | −1.335068006 | −1.915662244 | 1.412382482 |
| hsa-miR-376b | MI0002466 | −1.462115054 | −2.387333306 | 1.42532665 |
| hsa-miR-455-5p | MIMAT0003150 | −1.464923651 | −1.089434412 | 1.018810011 |
| hsa-miR-154* | MIMAT0000453 | −1.365192965 | −1.427770028 | 1.114498727 |
| hsa-miR-369-5p | MIMAT0001621 | −1.192026924 | −3.699726936 | 1.084192985 |
| hsa-miR-379 | MI0000787 | −1.461968244 | −1.164830838 | 1.306786907 |
| hsa-miR-508-3p | MIMAT0002880 | −1.007436551 to | −3.470796039 | 2.426909655 |
| (control) | | −0.262303581 | | |
| hsa-miR-507 | MI0003194 | −0.978310779 to | −3.536678217 | 2.446072063 |
| (control) | | −0.221168598 | | |
| hsa-miR-506 | MI0003193 | −1.023543855 to | −3.778310128 | 2.095831674 |
| (control) | | −0.165472432 | | |
| hsa-miR-510 | MI0003197 | −0.915219951 to | −3.738786844 | 1.973102546 |
| (control) | | −0.175580378 | | |
| hsa-miR-487a | MI0002471 | −1.483262811 to | −3.896097466 | 1.185404126 |
| (control) | | −0.244660874 | | |
| Class Ov2B | | | | |
| hsa-miR-502-5p | MIMAT0002873 | −1.073172242 | −1.573036604 | 0.91931496 |
| hsa-miR-652 | MI0003667 | −1.294366302 | 0.060957882 | 0.761730405 |
| hsa-miR-532-3p | MIMAT0004780 | −1.207007579 | 0.04586731 | 0.965366426 |
| hsa-miR-502-3p | MIMAT0004775 | −1.203200504 | −0.106090502 | 0.889469367 |
| hsa-miR-500* | MIMAT0002871 | −1.197728338 | 0.015163557 | 0.896929285 |
| (now goes by | | | | |
| hsa-miR-500a- | | | | |
| 3p) | | | | |
| hsa-miR-188-3p | MIMAT0004613 | −1.155787317 | −4.565222643 | 1.013228206 |
| hsa-miR-362-5p | MIMAT0000705 | −1.118053571 | 1.069380433 | 0.938564202 |
| hsa-miR-222 | MI0000299 | −1.398563949 | 2.643868139 | 1.251776602 |
| hsa-miR-501-3p | MIMAT0004774 | −1.336038298 | −1.051236291 | 0.941476517 |
| hsa-miR-34c-3p | MIMAT0004677 | −1.38080764 to | −1.021683048 | 2.217745802 |
| (control) | | 0.509145105 | | |
| hsa-miR-33a | MI0000091 | −1.151600279 to | −0.794541132 | 1.052283143 |
| (control) | | 0.883835041 | | |
| hsa-miR-660 | MI0003684 | −2.718832324 to | 2.116552559 | 1.005015061 |
| (control) | | −0.319646558 | | |
| hsa-miR-532-5p | MIMAT0002888 | −3.822049207 to | 1.786390032 | 0.91291073 |
| (control) | | −1.047596325 | | |
| hsa-miR-145 | MI0000461 | −1.222684829 to | 3.10252339 | 1.344452316 |
| (control) | | 1.417571694 | | |

The present invention identifies four unique classes described by microRNA signatures (Tables 1-2) whose expression in tumors is predictive of survival differences and patient response in ovarian cancer. Classes Ov1A and Ov1B displayed enrichment of specific microRNAs whose expression is elevated beyond the established benchmark and a phenotype with significantly poorer prognosis with cisplatin/carboplatin plus taxol therapy. Patients with tumors exhibiting these signatures should receive more aggressive initial therapy. Class Ov2A is indicative of an improved prognosis characterized by signature microRNA expression levels which are reduced below the established benchmark. Patients with tumors exhibiting this signature should receive initial therapy with cisplatin/carboptatin+taxol. Class Ov2B represents a fourth unique group that correlates poor prognosis with a signature of microRNA expression levels which are reduced below the established benchmark. Patients with tumors exhibiting this signature should receive more aggressive initial therapy. Finally, patients with tumors that exhibit the Ov3 signature should be placed on more aggressive initial therapy.

### Example 3

### MicroRNAs and Improvement of Survival in Glioblastoma Multiforme

In the performance of an assay, tissue from fresh frozen or FFPE primary tumors from glioblastoma multiforme patients who are to be treated with temozolomide/temodol therapy is obtained. MicroRNA is extracted using a small RNA extraction kit or other methods known in the art and expression is quantified using a method such as qRTPCR, microarray, next generation sequencing technologies, or flow cytometer. Control microRNAs as measured along with the combination of microRNAs whose expression level define the signature for a class.

Analysis of MicroRNAs in primary glioblastoma multiforme tumors from patients to be treated with temodol/temozolomide yielded a distinct class, denoted Class G1A, consisting of fourteen microRNAs whose expression define its signature. FIG. 7 represents the survival plot of patients having tumors that exhibit the Class G1A signature (dotted line) compared with the remaining patients (solid line). The significant separation defines a distinct class of patients with improved prognosis and a positive response to temodol/temozolomide therapy that is indicated by a signature defined by an elevated level of expression which exceeds the benchmark (specified in Table 3) of the microRNAs. of Class G1A. The low Kaplan-Meier survival p-value (0.0002) confirms that these fourteen microRNAs are up-regulated in glioblastoma patients with improved prognosis and response to therapy.

Additional analysis of primary glioblastoma multiforme tumors from patients to be treated with temodol/temozolomide identified two additional classes of microRNAs: Class G2A, whose signature consists of the expression levels of 15 microRNAs, and Class G2B, whose signature consists of the expression levels of 14 microRNAs. FIG. 8 depicts the survival plot of patients with tumors exhibiting the signature of Class G2A (dotted line) versus the remaining patients (solid line). The significant separation defines a distinct class of patients with improved prognosis and a positive response to temodol/temozolomide chemotherapy, indicated by a signature defined by a reduced level of expression which falls below the benchmark (specified in Table 3) of the microRNA from Classes G2A and G2B in their tumors. The low p-value (4.5E-05) indicates that these fifteen microRNAs are down-regulated in patients with improved prognosis.

Additionally, FIG. 9 shows a similar result, this time comparing patients treated with temodol/temozolomide who have tumors that exhibit the signature expression of microRNAs of Class G2B (dotted line) compared to the remaining patients (solid line). The significant separation defines a distinct class of patients with poorer prognosis and a poor response to temodol/temozolomide therapy. This is indicated by the signature defined by the reduced level of expression which falls below the benchmark (specified in Table 3) of the microRNA signature from this class. The p-value is again low (0.0087), indicating that negative regulation of these microRNAs in glioblastoma multiforme tumors is correlated with a poorer prognosis and response to temodol/temozolomide therapy. Table 3 below lists the microRNAs from Classes G1A, G2A.

### Table 3—MicroRNA Signatures Predictive of Response to Therapy.

MicroRNA Signatures which are Predictive of Response to Temodol/Temozolomide.

The signature expression of microRNAs in Class G1A are elevated above the benchmark in the longer survivors. The signature of the microRNAs in Class G2A displays repressed expression below the benchmark in the longer survivors. In the signature of Class G2B, the microRNAs in class G2B display repressed expression below the benchmark in patients with poor survival. For Class G1A, a binary subscore of a microRNA is assigned a score of 1 if the assayed expression level is greater than the benchmark and a score of 0 if the assayed expression level is lower than the benchmark. For Classes G2A and G2B, a binary subscore of a microRNA is assigned a score of 1 if the assayed expression level is less than the benchmark and a score of 0 if the assayed expression level is greater than the benchmark. The “norm factors” are specifically calculated numeric representations by which patient data needs to be normalized. “Norm factor 1” shall be subtracted from the patient assayed expression level. Subsequently, this total will be divided by “Norm factor 2” to produce normalized expression values.

TABLE 3

| | miRBase | | | |
|---|---|---|---|---|
| | Accession | | | |
| | (v20) | Benchmark | Norm factor 1 | Norm Factor 2 |
| Class G1A | | | | |
| hsa-miR-130a | MI0000448 | 1.231227226 | 3.325408769 | 0.667094721 |
| hsa-miR-130b | MI0000748 | 1.295839364 | 1.131446206 | 0.918317408 |
| hsa-miR-140-5p | MIMAT0000431 | 0.981890354 | 0.558897171 | 1.218449637 |
| hsa-miR-17-3p | MIMAT0000071 | 0.70485791 | −0.673960356 | 1.763309038 |
| hsa-miR-17-5p | MIMAT0000070 | 1.21188306 | 1.551532007 | 0.888624944 |
| hsa-miR-181a-5p | MIMAT0000256 | 1.301579822 | 3.032404442 | 0.71900205 |
| hsa-miR-181a-3p | MIMAT0000270 | 0.931773074 | −1.35233826 | 1.634662587 |
| hsa-miR-181b-1 | MI0000270 | 1.273869181 | 2.975499085 | 0.662751848 |
| hsa-miR-181d | MI0003139 | 1.143099938 | 1.673723083 | 0.799548561 |
| hsa-miR-186 | MI0000483 | 1.376875527 | −0.112755307 | 0.697436864 |
| hsa-miR-340 | MI0000802 | 0.822607511 | −3.312042611 | 2.605942459 |
| hsa-miR-361-5p | MIMAT0000703 | 1.244064741 | 1.612031941 | 0.506480095 |
| hsa-miR-454-3p | MIMAT0003885 | 0.833872727 | −2.456643159 | 2.363463048 |
| hsa-miR-92 (now | MIMAT0000092 | 1.439728529 | 3.499270184 | 0.780803906 |
| goes by hsa-miR- | | | | |
| 92a-3p) | | | | |
| hsa-miR-219-5p | MIMAT0000276 | −0.466398547 to | 1.656478264 | 2.521357367 |
| (control) (now | | 0.791861378 | | |
| goes by hsa-miR- | | | | |
| 219a-5p) | | | | |
| hsa-miR-532-5p | MIMAT0002888 | −0.095306091 to | −2.074825365 | 2.159962041 |
| (control) | | 1.364586424 | | |
| hsa-miR-301a | MI0000745 | −0.141301126 to | 0.002598053 | 2.155833527 |
| (control) | | 1.25885431 | | |
| hsa-miR-491 | MI0003126 | −0.358025554 to | −2.200228883 | 2.052944855 |
| (control) | | 1.323629779 | | |
| hsa-miR-224 | MI0000301 | −0.993342054 to | −8.347916287 | 3.150041159 |
| (control) | | 0.388836661 | | |
| Class G2A | | | | |
| hsa-miR-132 | MI0000449 | −1.083720972 | 0.162728947 | 0.807924392 |
| hsa-miR-142-3p | MIMAT0000434 | −1.139399656 | 2.694705049 | 1.366544653 |
| hsa-miR-148a | MI0000253 | −0.997861307 | −0.124330204 | 2.462957624 |
| hsa-miR-155 | MI0000681 | −1.254804151 | 0.72997553 | 0.951690309 |
| hsa-miR-193a | MI0000487 | −0.832866559 | −0.776177045 | 1.668179621 |
| hsa-miR-202 | MI0003130 | −1.372414449 | 1.96404948 | 0.738762376 |
| hsa-miR-221 | MI0000298 | −0.998167582 | 0.28138897 | 1.766998804 |
| hsa-miR-222 | MI0000299 | −1.193753746 | 2.625511946 | 1.55786209 |
| hsa-miR-223 | MI0000300 | −1.327889968 | 1.888630002 | 1.044665015 |
| hsa-miR-25 | MI0000082 | −1.215746336 | 2.710785579 | 0.778174653 |
| hsa-miR-34b* | MIMAT0000685 | −1.356294527 | −1.13987101 | 2.497842462 |
| (now goes by | | | | |
| hsa-miR-34b-5p) | | | | |
| hsa-miR-451 | MI0001729 | −1.228496895 | 3.069318398 | 1.425430985 |
| hsa-miR-487a | MI0002471 | −0.745618978 | −0.71668969 | 1.653339715 |
| hsa-miR-487b | MI0003530 | −1.293493059 | 2.000747795 | 0.690829198 |
| hsa-miR-509 | MI0003196 | −0.961479682 | −0.383610688 | 1.238096317 |
| hsa-miR-10b | MI0000267 | 0.324058533 to | 0.159281513 | 3.342586596 |
| (control) | | 0.886701561 | | |
| hsa-miR-299-5p | MIMAT0002890 | 0.084938104 to | −3.371655666 | 3.045087761 |
| (control) | | 0.755216406 | | |
| hsa-miR-374a | MI0000782 | 0.003614354 to | 0.108384996 | 1.917530026 |
| (control) | | 0.724296135 | | |
| hsa-miR-26a | MI0000083 | −0.11539484 to | 4.968952701 | 1.168625217 |
| (control) | | 0.621417647 | | |
| hsa-miR-338 | MI0000814 | 0.051151757 to | 1.480156311 | 2.069457465 |
| (control) | | 0.758635576 | | |
| Class G2B | | | | |
| hcmv-miR-US25-1 | MI0001684 | −1.568630522 | −5.363107938 | 3.281281222 |
| hsa-miR-133b | MI0000822 | −1.629989273 | −5.242573422 | 3.34676077 |
| hsa-miR-141 | MI0000457 | −1.2937725 | −4.988309618 | 2.970795185 |
| hsa-miR-205 | MI0000285 | −1.473876203 | −6.476429347 | 3.040412962 |
| hsa-miR-423 | MI0001445 | −1.362248859 | −4.869558577 | 2.789634818 |
| hsa-miR-425-3p | MI0001448 | −1.648434776 | −6.129999428 | 2.957446863 |
| hsa-miR-488* | MI0003123 | −1.526301741 | −6.274312455 | 2.804855219 |
| hsa-miR-490-3p | MI0003125 | −1.394878946 | −6.780636418 | 3.143941247 |
| hsa-miR-516b-5p | MIMAT0002859 | −1.387476897 | −6.161366976 | 3.485364771 |
| hsa-miR-517* | MIMAT0002851 | −1.201767859 | −8.29159141 | 2.507559868 |
| (now goes by | | | | |
| hsa-miR-517-5p) | | | | |
| hsa-miR-654-5p | MI0003676 | −1.732447408 | −5.150937671 | 3.072252009 |
| hsa-miR-767-5p | MIMAT0003882 | −1.525308252 | −6.280307112 | 3.148543051 |
| kshv-miR-K12-7 | MI0002479 | −1.378925539 | −6.258901548 | 3.414301381 |
| hsa-miR-191* | MIMAT0001618 | −1.736317622 to | −4.93287025 | 4.470521766 |
| (control) (now | | −1.373878662 | | |
| hsa-191-3p) | | | | |
| hsa-miR-563 | MI0003569 | −0.971892958 to | −8.366770556 | 4.371779634 |
| (control) | | −0.603815151 | | |
| hsa-miR-662 | MI0003670 | −1.893167776 to | −6.146801647 | 3.398947174 |
| (control) | | −1.416648287 | | |
| hsa-miR-518b | MI0003156 | −1.150384714 to | −8.7449684 | 3.357742052 |
| (control) | | −0.670460385 | | |
| hsa-miR-371-3p | MIMAT0000723 | −1.458687081 to | −7.81594995 | 3.289513218 |
| (control) (now | | −0.965437265 | | |
| goes by hsa-miR- | | | | |
| 371a-3p) | | | | |

The present invention identifies three novel Classes with microRNA signatures for use in diagnostics and determining treatments for patients with glioblastoma multiforme tumors. The expression levels of these microRNAs define signatures that are predictive of survival differences and response of patients with glioblastoma multiforme tumors when treated with temodol/temozolomide. Class G1A displayed enrichment of specific signature microRNAs whose, expression, when elevated above the benchmark, define a signature indicative of a Class of patients with significantly improved prognosis and a positive response to temodol/temozolomide therapy.

Furthermore, expression of microRNAs of Class G2A define a signature that is indicative of an improved prognosis and response to temodol/temozolomide therapy when these microRNAs have repressed expression below the benchmark. These two groups of patients can have positive response to temodol/temozolomide. Finally, Class G2B represents a third unique group that shows poor prognosis and response to therapy when its microRNA expression levels exhibit repressed expression below the “benchmark”. Patients with tumors that exhibit this signature should receive a more aggressive initial treatment.

The present invention, as previously described in Example 1, discloses an algorithm that is used to determine, for each patient, a PScore for each Class. Similar to the ovarian cancer example, in order to compare the assayed values with the established standards outlined in Table 3, the controls will be normalized to benchmark control values established for each class. For Class G1A, the assayed negative control will be normalized to a benchmark of −9.369486843 and the assayed positive control will be normalized to a benchmark of 10.58611861. For Class G2A, the assayed negative control will be normalized to a benchmark of −9.123762714 and the assayed positive control will be normalized to a benchmark of 10.66344757. For Class G2B, the assayed negative control will be normalized to a benchmark of −11.48771791 and the assayed positive control will be normalized to a benchmark of 10.11775335. For Class G1A, the subscore of a microRNA is a “1” if the assayed expression level is greater than the benchmark specified in Table 3. Should this condition not be fulfilled, the subscore for the microRNA is “0”. For Classes G2A and G2B, the assayed expression level of a microRNA must be lower than the benchmark also specified in Table 3 in order to attain a subscore of “1”. Should this condition not be fulfilled, the subscore for the microRNA is “0”.

### Example 4

### Description of MicroRNA Signatures for Ovarian Cancer and Glioblastoma Multiforme

The microRNAs indicative of each class have been generated based on the expression levels of the microRNAs, their signatures, and their empirical association with particular prognoses. The signatures accurately predict cancer patient response to the chemotherapy regimen of which they were based as follows:

### Ovarian Cancer Response to Cisplatin/Carboplatin+Taxol Chemotherapy:

Signature of Class Ov1A: MicroRNA's which Predict Poor Prognosis when the Signature microRNAs are Elevated Above the Benchmark.
hsa-miR-33b
hsa-miR-30d
hsa-miR-30d-3p
hsa-miR-370
hsa-miR-934
hsa-miR-519e-5p
hsa-miR-30b*
hsa-miR-663a
hsa-miR-583
hsa-miR-526b
hsa-miR-9-5p (control)
hsa-miR-9-3p (control)
hsa-miR-501-5p (control)
hsa-miR-488 (control)
hsa-miR-144* (control)
Signature of Class Ov1B: MicroRNA's which Predict Poor Prognosis when the Signature microRNAs are Elevated Above the Benchmark.
hsa-miR-136
hsa-miR-337-5p
hsa-miR-377
hsa-miR-381
hsa-miR-376a-1
hsa-miR-379 (control)
hsa-miR-411 (control)
hsa-miR-299-3p (control)
hsa-miR-154 (control)
hsa-miR-376c (control)
Signature of Class Ov2A: MicroRNA's which Predict Good Prognosis when the Signature microRNAs are Repressed Below the Benchmark.
hsa-miR-136
hsa-miR-377
hsa-miR-410
hsa-miR-376b
hsa-miR-455-5p
hsa-miR-154*
hsa-miR-369-5p
hsa-miR-379
hsa-miR-508-3p (control)
hsa-miR-507 (control)
hsa-miR-506 (control)
hsa-miR-510 (control)
hsa-miR-487a (control)
Signature of Class Ov2B: MicroRNA's which Predict Poor Prognosis when the Signature microRNAs are Repressed Below the Benchmark.
hsa-miR-502-5p
hsa-miR-652
hsa-miR-532-3p
hsa-miR-502-3p
hsa-miR-500a-3p
hsa-miR-188-3p
hsa-miR-362-5p
hsa-miR-222
hsa-miR-501-3p
hsa-miR-34c-3p (control)
hsa-miR-33a (control)
hsa-miR-660 (control)
hsa-miR-532-5p (control)
hsa-miR-145 (control)
Signature of Class Ov3: MicroRNA's which Predict Poor Prognosis when the Signature microRNAs are Elevated Above the Benchmark.
hsa-miR-377
hsa-miR-381
hsa-miR-376a-1
hsa-miR-410
hsa-miR-379 (control)
hsa-miR-411 (control)
hsa-miR-299-3p (control)
hsa-miR-154 (control)
hsa-miR-376c (control)

### Glioblastoma Multiforme Response to Temodol/Temozolomide Chemotherapy:

Signature of Class G1A: MicroRNA's which Predict Good Prognosis when the Signature microRNAs are Elevated Above the Benchmark.
hsa-miR-130a
hsa-miR-130b
hsa-miR-140-5p
hsa-miR-17-3p
hsa-miR-17-5p
hsa-miR-181a-5p
hsa-miR-181a-3p
hsa-miR-181b-1
hsa-miR-181d
hsa-miR-186
hsa-miR-340
hsa-miR-361-5p
hsa-miR-454-3p
hsa-miR-92a-3p
hsa-miR-219a-5p (control)
hsa-miR-532-5p (control)
hsa-miR-301a (control)
hsa-miR-491 (control)
hsa-miR-224 (control)
Signature of Class G2A: MicroRNA's which Predict Good Prognosis when the Signature microRNAs are Repressed Below the Benchmark.
hsa-miR-132
hsa-miR-142-3p
hsa-miR-148a
hsa-miR-155
hsa-miR-193a
hsa-miR-202
hsa-miR-221
hsa-miR-222
hsa-miR-223
hsa-miR-25
hsa-miR-34b-5p
hsa-miR-451
hsa-miR-487a
hsa-miR-487b
hsa-miR-509
hsa-miR-10b (control)
hsa-miR-299-5p (control)
hsa-miR-374a (control)
hsa-miR-26a (control)
hsa-miR-338 (control)
Signature of Class G2B: MicroRNA's which Predict Poor Prognosis when the Signature microRNAs are Repressed Below the Benchmark.
hcmv-miR-US25-1
hsa-miR-133b
hsa-miR-141
hsa-miR-205
hsa-miR-423
hsa-miR-425-3p
hsa-miR-488*
hsa-miR-490-3p
hsa-miR-516b-5p
hsa-miR-517-5p
hsa-miR-654-5p
hsa-miR-767-5p
kshv-miR-K12-7
hsa-miR-191-3p (control)
hsa-miR-563 (control)
hsa-miR-662 (control)
hsa-miR-518b (control)
hsa-miR-371a-3p (control)

### Example 5

### Description of Use of Signatures

The microRNA signatures disclosed herein enable clinical treatment of cancer through the design and development of a diagnostic and a method of determining an appropriate therapy. Each diagnostic will predict patient response to a standard therapy, allowing for:

Identification of patients with tumors that will respond to cisplatin/carboplatin plus taxol therapy,
Identification of patients with tumors that will respond to temedol/temozolmide therapy,
More aggressive treatment of predicted non-responders.
Placement of predicted non-responders in clinical trials prior to failure of standard therapy.
Prevent patients predicted to respond positively to standard therapy from entering unnecessary clinical trials.

### Example 6

### Kits

A kit can be assembled to use a qRT-PCR based method of measuring the level of expression of the signature microRNAs in a sample, the use of a custom microRNA microarray that assays the level of expression of the signature microRNAs or to use a microRNA sequencing technique to measure the expression level of the signature microRNAs. Building a custom microRNA microarray using a distinct set of microRNAs complementary to the signatures associated with positive and negative prognoses can allow one to easily assay the microRNA expression levels and compare them to the microRNA signatures associated with the prognoses. Kits could also include control microRNAs to compare the individual assay results to other instances of conducting the assay and between patients. Kits can include all reagents needed to perform the assays. They can be designed to be used with various types of equipment for PCR, array hybridization, sequencing, data collection, etc., as appropriate.

The invention further concerns a kit comprising one or more of (1) extraction buffer/reagents and protocol; (2) reverse transcription buffer/reagents and protocol; and (3) qPCR buffer/reagents and protocol suitable for performing any of the foregoing methods.

### Example 7

### Embodiments of the Invention

Particular embodiments of the invention are described.

A method of improving the clinical outcome for human patients diagnosed with Ovarian cancer when treated with platinum based chemotherapy, comprising,

obtaining samples of ovarian cancer cells from the patients,
determining the expression levels of Class Ov1A microRNAs:
hsa-miR-33b
hsa-miR-30d
hsa-miR-30d-3p
hsa-miR-370
hsa-miR-934
hsa-miR-519e-5p
hsa-miR-30b*
hsa-miR-663a
hsa-miR-583
hsa-miR-526b
in each sample,
determining whether the expression levels of said microRNAs are above or below the benchmark for each microRNA,
calculating a PScore for each patient, and
treating patients having PScore of greater or equal to 0.50 with a standard platinum based chemotherapy.
A method of improving the clinical outcome for human patients diagnosed with Ovarian cancer when treated with platinum based chemotherapy, comprising,
obtaining samples of ovarian cancer cells from the patients,
determining the expression levels of Class Ov1B microRNAs:
hsa-miR-136
hsa-miR-337-5p
hsa-miR-377
hsa-miR-381
hsa-miR-376a-1
in each sample,
determining whether the expression levels of said microRNAs are above or below the benchmark for each microRNA,
calculating a PScore for each patient, and
treating patients having PScore of greater or equal to 0.50 with a standard platinum based chemotherapy.
A method of improving the clinical outcome for human patients diagnosed with Ovarian cancer when treated with platinum based chemotherapy, comprising,
obtaining samples of ovarian cancer cells from the patients,
determining the expression levels of Class Ov2A microRNAs:
hsa-miR-136
hsa-miR-377
hsa-miR-410
hsa-miR-376b
hsa-miR-455-5p
hsa-miR-154*
hsa-miR-369-5p, and
hsa-miR-379,
in each sample,
determining whether the expression levels of said microRNAs are above or below the benchmark for each microRNA,
calculating a PScore for each patient, and
treating patients having PScore of greater or equal to 0.50 with a therapy that is more aggressive than standard platinum based chemotherapy.
A method of improving the clinical outcome for human patients diagnosed with Ovarian cancer when treated with platinum based chemotherapy, comprising,
obtaining samples of ovarian cancer cells from the patients,
determining the expression levels of Class Ov2B microRNAs:
hsa-miR-502-5p
hsa-miR-652
hsa-miR-532-3p
hsa-miR-502-3p
hsa-miR-500a-3p
hsa-miR-188-3p
hsa-miR-362-5p
hsa-miR-222
hsa-miR-501-3p
in each sample,
determining whether the expression levels of said microRNAs are above or below the benchmark for each microRNA,
calculating a PScore for each patient, and
treating patients having PScore of greater or equal to 0.50 with a standard platinum based chemotherapy.

A method of improving the clinical outcome for human patients diagnosed with Ovarian cancer when treated with platinum based chemotherapy, comprising,

obtaining samples of ovarian cancer cells from the patients,
determining the expression levels of Class Ov3 microRNAs:
hsa-miR-377
hsa-miR-381
hsa-miR-376a-1
hsa-miR-410
in each sample,
determining whether the expression levels of said microRNAs are above or below the benchmark for each microRNA,
calculating a PScore for each patient, and
treating patients having PScore of greater or equal to 0.50 with a standard platinum based chemotherapy.
A method of improving the clinical outcome for human patients diagnosed with Glioblastoma multiforme when treated with Temodol/Temozolomide chemotherapy, comprising:
obtaining samples of glioblastoma multiforme cancer cell from the patients,
determining the expression levels of G1A microRNAs:
hsa-miR-130a
hsa-miR-130b
hsa-miR-140-5p
hsa-miR-17-3p
hsa-miR-17-5p
hsa-miR-181a-5p
hsa-miR-181a-3p
hsa-miR-181b-1
hsa-miR-181d
hsa-miR-186
hsa-miR-340
hsa-miR-361-5p
hsa-miR-454-3p
hsa-miR-92a-3p,
in each sample,
determining whether the expression levels of said microRNAs are above or below the benchmark for each microRNA,
calculating a PScore for each patient, and
treating patients having PScore of equal to or greater than 0.5 with Temodol/Temozolomide based chemotherapy.

A method of improving the clinical outcome for human patients diagnosed with Glioblastoma multiforme when treated with Temodol/Temozolomide chemotherapy, comprising:

obtaining samples of glioblastoma multiforme cancer cell from the patients,
determining the expression levels of G2A microRNAs:
hsa-miR-132
hsa-miR-142-3p
hsa-miR-148a
hsa-miR-155
hsa-miR-193a
hsa-miR-202
hsa-miR-221
hsa-miR-222
hsa-miR-223
hsa-miR-25
hsa-miR-34b-5p
hsa-miR-451
hsa-miR-487a
hsa-miR-487b
hsa-miR-509
in each sample,
determining whether the expression levels of said microRNAs are above or below the benchmark for each microRNA,
calculating a PScore for each patient, and
treating patients having PScore of equal to or greater than 0.5 with Temodol/Temozolomide based chemotherapy.
A method of improving the clinical outcome for human patients diagnosed with Glioblastoma multiforme when treated with Temodol/Temozolomide chemotherapy, comprising:
obtaining samples of glioblastoma multiforme cancer cell from the patients,
determining the expression levels of G2B microRNAs:
hcmv-miR-US25-1
hsa-miR-133b
hsa-miR-141
hsa-miR-205
hsa-miR-423
hsa-miR-425-3p
hsa-miR-480*
hsa-miR-490-3p
hsa-miR-516b-5p
hsa-miR-517-5p
hsa-miR-526c
hsa-miR-654-5p
hsa-miR-767-5p
kshv-miR-K12-7,
in each sample,
determining whether the expression levels of said microRNAs are above or below the benchmark for each microRNA,
calculating a PScore for each patient, and
treating patients having PScore of greater than or equal to 0.5 with a therapy that is more aggressive than standard Temodol/Temozolomide based chemotherapy.

A method of improving the clinical outcome for human patients diagnosed with Ovarian cancer when treated with platinum based chemotherapy, comprising,

obtaining samples of ovarian cancer cells from the patients,
determining the expression levels of microRNAs of Classes of microRNA selected from the group comprising Ov1A, Ov1B, Ov2A, Ov2B, Ov3 microRNAs in each sample,
determining whether the expression levels of said microRNAs are above or below the benchmark for each microRNA,
calculating a PScore for each patient, and
treating patients having PScore of greater than or equal to 0.5 for Ov1A microRNAs with a therapy that is more aggressive than standard platinum based chemotherapy,
treating patients having PScore of greater than or equal to 0.5 for Ov1B microRNAs with a therapy that is more aggressive than standard platinum based chemotherapy,
treating patients having PScore of greater than or equal to 0.5 for Ov2A microRNAs with a standard platinum based chemotherapy,
treating patients having PScore of greater than or equal to 0.5 for Ov2B microRNAs with a therapy that is more aggressive than standard platinum based chemotherapy, and
treating patients having PScore of greater than or equal to 0.5 for Ov3 microRNAs with a therapy that is more aggressive than standard platinum based chemotherapy.
A method of improving the clinical outcome for human patients diagnosed with Gliblastoma multiforme when treated with Temodol/Temozolomide based chemotherapy, comprising,
obtaining samples of Glioblastoma multiforme cancer cells from the patients,
determining the expression levels of microRNAs of Classes of microRNA selected from the group comprising G1A, G2A and G2B microRNAs in each sample,
determining whether the expression levels of said microRNAs are above or below the benchmark for each microRNA,
calculating a PScore for each patient, and
treating patients having PScore of greater than or equal to 0.5 for G1A, microRNAs with a standard Temodol/Temozolomide based chemotherapy,
treating patients having PScore of greater than or equal to 0.5 for G2A, microRNAs with a standard Temodol/Temozolomide based chemotherapy,
treating patients having PScore of greater than or equal to 0.05 for G2B, and microRNAs with a therapy that is more aggressive than standard Temodol/Temozolomide based chemotherapy.

A microarray chip having only sequences complementary to a the microRNAs selected from the group of Ov1A, Ov1B, Ov2A, Ov2B, Ov3, G1A, G2A and G2B microRNAs and appropriate control sequences.

A kit for measuring the level of expression of the microRNAs selected from the group of Ov1A, Ov1B, Ov2A, Ov2B, Ov3, G1A, G2A and G2B microRNAs.

In any of the above embodiments, microRNA expression levels can be determined using methods known in the art or that may become available for those of skill in the art. These methods can include the use of microarray chips, flow cytometry, sequencing and various PCR techniques.

### BIBLIOGRAPHY

Lu J, Getz G, Miska E A, Alvarez-Saavedra E, Lamb J, Peck D, Sweet-Cordero A, Ebert B L, Mak R H, Ferrando A A et al: MicroRNA expression profiles classify human cancers. Nature 2005, 435(7043):834-838.
2. Iorio M V, Ferracin M, Liu C G, Veronese A, Spizzo R, Sabbioni S, Magri E, Pedriali M, Fabbri M, Campiglio M et al: MicroRNA gene expression deregulation in human breast cancer. Cancer Res 2005, 65(16):7065-7070.
3. Volinia S, Cahn G A, Liu C G, Ambs S, Cimmino A, Petrocca F, Visone R, Iorio M, Roldo C, Ferracin M et al: A microRNA expression signature of human solid tumors defines cancer gene targets. Proc Natl Acad Sci USA 2006, 103(7):2257-2261.
4. Cahn G A, Dumitru C D, Shimizu M, Bichi R, Zupo S, Noch E, Aldler H, Rattan S, Keating M, Rai K et al: Frequent deletions and down-regulation of micro-RNA genes miR15 and miR16 at 13q14 in chronic lymphocytic leukemia. Proc Natl Acad Sci USA 2002, 99(24):15524-15529.
5. Cahn G A, Ferracin M, Cimmino A, Di Leva G, Shimizu M, Wojcik S E, Iorio M V, Visone R, Sever N I, Fabbri M et al: A MicroRNA signature associated with prognosis and progression in chronic lymphocytic leukemia. N Engl J Med 2005, 353(17):1793-1801.
6. Yanaihara N, Caplen N, Bowman E, Seike M, Kumamoto K, Yi M, Stephens R M, Okamoto A, Yokota J, Tanaka T et al: Unique microRNA molecular profiles in lung cancer diagnosis and prognosis. Cancer Cell 2006, 9(3):189-198.

What is claimed is:

1. A method of classifying human patients diagnosed with Ovarian cancer, comprising,
obtaining samples of ovarian cancer cells from the patients,
determining the expression levels of microRNA Classes selected from the group comprising Ov1A, Ov1B, Ov2A, Ov2B, Ov3 microRNAs in each sample,
determining whether the expression levels of each of said microRNAs are above or below the benchmark for each microRNA,
calculating a PScore for each patient, and
classifying patients having PScore of greater than or equal to 0.5 for a Class of microRNA as patients with tumors exhibiting the signature of that Class.

2. The method of claim 1 where the expression levels are determined by a technique selected from the group comprising PCR, RT-PCR, microarray hybridization and flow cytometry.

3. A method of classifying human patients diagnosed with Glioblastoma multiforme, comprising,
obtaining samples of ovarian cancer cells from the patients,
determining the expression levels of microRNA Classes selected from the group comprising G1A, G2A and G2B microRNAs in each sample,
determining whether the expression levels of each of said microRNAs are above or below the benchmark for each microRNA,
calculating a PScore for each patient, and
classifying patients having PScore of greater than or equal to 0.5 for a Class of microRNA as patients with tumors exhibiting the signature of that Class.

4. The method of claim 3 where the expression levels are determined by a technique selected from the group comprising PCR, RT-PCR, microarray hybridization and flow cytometry.

5. A kit for measuring the level of expression of the microRNAs selected from the group of OV1A, OV1B, OV2A, OV2B, Ov3, G1A, G2A and G2B microRNAs.

6. The kit of claim 5 where the expression levels are determined by a technique selected from the group comprising PCR, RT-PCR, microarray hybridization and flow cytometry.
